(19) United States
(12) Patent Application Publication
(10) Pub. No.: US 2013/0289588 A1
(43) Pub. Date: Oct. 31, 2013

(54) VASCULAR CLIP

(71) Applicants: Thomas P. Robertson, Athens, GA (US); Stephen P. Chelko, Winterville, GA (US)

(72) Inventors: Thomas P. Robertson, Athens, GA (US); Stephen P. Chelko, Winterville, GA (US)

(73) Assignee: UNIVERSITY OF GEORGIA RESEARCH FOUNDATION, INC., Athens, GA (US)

(21) Appl. No.: 13/996,162

(22) PCT Filed: Dec. 20, 2011
(86) PCT No.: PCT/US11/66088

§ 371 Date: Jun. 20, 2013

Related U.S. Application Data
(60) Provisional application No. 61/425,055, filed on Dec. 20, 2010.

(51) Int. Cl. A61B 17/122

(52) U.S. Cl. CPC A61B 17/122
USPC 606/158

(57) ABSTRACT

The present disclosure provides a clip for application to a renal artery to induce hypertension. The clip includes a pair of arms between which is formed a slot. The arms are supported by a base portion. The clip may include various curved surfaces to prevent snagging and damage when applied to the renal artery. Also, the clip is preferably constructed of stock titanium rods to have a unitary and stiff construction. Further, the clip includes a pair of cylindrical suture openings that retain a suture passed therethrough after the clip is applied to the renal artery. The suture firmly retains the clip on the artery.

### CROSS-REFERENCE TO PRIORITY APPLICATIONS

This application claims priority to U.S. Provisional Application No. 61/425,055 filed on Dec. 20, 2010 which is incorporated herein by reference in its entirety.

### ACKNOWLEDGEMENT OF GOVERNMENT SUPPORT

This invention was made with government support under 1R01NS054117-01A2 awarded by the National Institutes of Health. The government has certain rights in the invention.

### FIELD

This disclosure relates to implantable clips to induce hypertension in laboratory animals and, in particular, clips for the renal artery.

### BACKGROUND

Renal hypertension may be induced by applying a clip to a renal artery of a laboratory animal. For example, as shown in FIG. 1, a strip of malleable silver may be bent back on itself to pinch the renal artery. Such clips can effectively induce renal hypertension, but often have unpredictable results. Some percentage of rats, for example, die due to renal hypertension. In others, the amount of hypertension widely varies or may not even occur.

Leenen et al. studied a clip made from a 2 mm×32 mm×1.6 mm rectangular block of silver wherein a slot with a 2 mm depth was formed in the block. See, A Solid Silver Clip for Induction of Predictable Levels of Renal Hypertension in the Rat, Journal of Applied Physiology, Vol. 31, No. 1, July 1971, pp. 142-144. Varying widths of the slot (0.20, 0.25, 0.30 and 0.35 mm) produced somewhat more reliable hypertension of varying levels in inverse proportion to the width of the slot.

Despite the improvements observed with the Leenen et al. clip, there remains a need for greater consistency and reliability in the use of clips to induce renal hypertension in laboratory animals.

### SUMMARY

The present invention overcomes the problems of the prior art by providing, in one implementation, a vascular or renal artery clip including a pair of arms defining a slot, a base portion connecting the pair of arms and curved surfaces that minimize the risk of snagging. The clip may also or alternatively include a retainer mechanism that locks it on the vascular structure, such as a suture hole extending through the arms at an edge of the slot. Also, the clip may be constructed of a relatively stiff material, such as titanium, with a modulus of over 100 GPa so that the arms do not bend relative to the base portion. In another implementation, the clip is constructed out of titanium rod stock with a saw to slice away and form the slot of appropriate width.

These and other features and advantages of implementations of the present disclosure will become more readily apparent to those skilled in the art upon consideration of the following detailed description and accompanying drawings, which describe both the preferred and alternative implementations of the present disclosure.

### BRIEF DESCRIPTION OF THE DRAWINGS

FIG. 1 is a schematic of a prior art clip that is a bent strip of silver folded onto a renal artery;

FIG. 2 is an elevation view of a clip; and

FIG. 3 is a cross-section of the clip of FIG. 2.

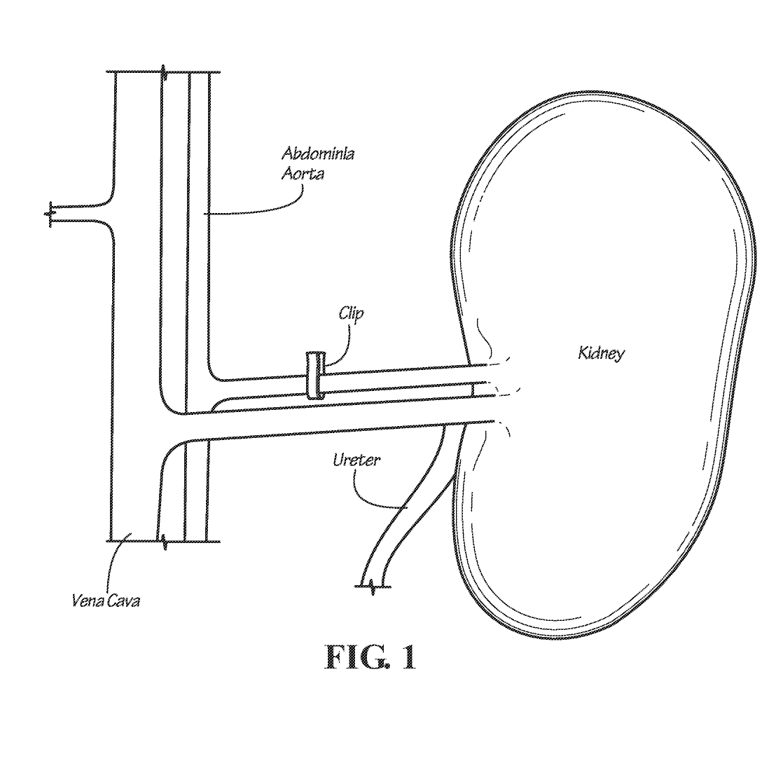

### DETAILED DESCRIPTION

The present disclosure now will be described more fully hereinafter. Indeed, these implementations can be embodied in many different forms and should not be construed as limited to the implementations set forth herein; rather, these implementations are provided so that this disclosure will satisfy applicable legal requirements. As used in the specification, and in the appended claims, the singular forms “a”, “an”, “the”, include plural referents unless the context clearly dictates otherwise. The term “comprising” and variations thereof as used herein is used synonymously with the term “including” and variations thereof and are open, non-limiting terms.

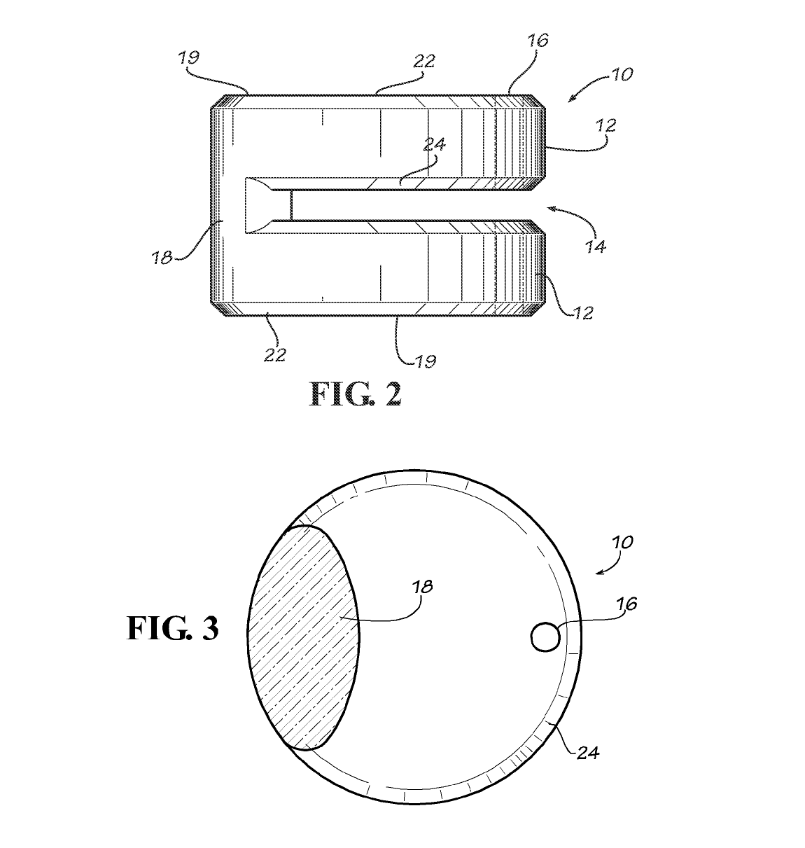

In FIG. 2, a clip 10 for application to a renal artery to induce hypertension is shown. The clip 10 includes a pair of arms 12 between which is formed a slot 14. The arms 12 are supported by a base portion 18. The clip 10 may include various curved surfaces to prevent snagging and damage when applied to the renal artery. Also, the clip 10 is preferably constructed of stock titanium rods to have a unitary and stiff construction. Further, the clip 10 includes a pair of cylindrical suture openings 16 that retain a suture passed therethrough after the clip is applied to the renal artery. The suture firmly retains the clip 10 on the artery.

It should be noted that implementations disclosed herein are described for use with a rat model, but could also be used for other animal models. Also, implementations of the clip may be used on humans for applications or treatments that may benefit from induced hypertension.

Referring again to FIG. 2, the clip 10 has a generally cylindrical shape into which is defined the slot 14. For example, stock titanium rod with a 0.118 inch or 4 mm diameter may be sliced into short lengths, e.g., 0.079 inch or 2 mm, to form lateral ends 19. The slot 14 is formed by sawing away a central portion of the rod to a desired depth. Formation of the slot 14, in turn, forms the arms 12 and base portion 18 of the clip 10. Generally, the illustrated implementation has a U-shape from one elevation view. The saw may be a 0.79 mm slitting saw. The edges of cuts may also be deburred using a 90 degree double-angle milling cutter.

Although titanium is disclosed in the illustrated implementation, other materials may be used, especially if they are sufficiently rigid to not bend or deform during surgical placement or everyday loads. For example, although silver is disclosed in the prior art, titanium has a significantly higher Young's modulus 100 to 110 GPa. Generally, anything higher than 83 MPa, however, would be an improvement in stiffness, avoiding bending of the clip 10 during installation. Notably, the use of the suture openings 16 or other securing mechanism enables the use of stiffer construction without the risk that the clip will fall off after implantation. This is unlike conventional clips that employ malleable silver to allow the clip to be deformed to lock onto the renal artery during implantation. At the same time, this malleability allows changes in the clip width with handling during surgery. Also, an implanted clip may open up and become dislodged.

Each of the arms 12 has a cylindrical shape when viewed axially, such as in FIG. 3, due to creation from the rod stock. The arms are also similarly sized and shaped and spaced from the midline of the slot 14. Thus the clip 10 may be symmetrically shaped about the midline of the slot.

Defined on the lateral circular edge of each of the arms is a lateral chamfer 22 that reduces the likelihood of sharp edges injuring the lab animal during, or after, implantation. For instance, the chamfer may be 0.084 inch×45 degrees. This chamfer eliminates the right angle between the lateral surface and cylindrical surface of the arms 12. In other words, the chamfer surface is at a 135 degree angle with respect to the lateral surface and cylindrical surface of the arms 12. Advantageously, the illustrated implementation has no two external surfaces intersecting at 90 degree or smaller angles. Alternatively stated, the outer surfaces are fashioned to have obtuse angles.

The arms could also be elliptical, square, triangular or irregular shapes depending upon a range of factors like the size of the animal, cost or ease of installation. Circular in the illustrated implementation does have the advantage, however, of reducing edges and snagging.

Each of the arms 12 also includes a medial chamfer 24 that extends through the partial arc left by defining the slot 14. The medial chamfer is also a 0.084 inch×45 degree chamfer in the implementation of FIG. 3. It may also be varied as described above, but regardless of size or angle, has some advantage in that it eliminates the right angle between the outer circumferential surface and the medial surface of the arms 12 to prevent or reduce snagging or injury during implanting.

The base portion 18, as shown in FIG. 2, extends between the arms 12 and includes an external peripheral surface that is part of the cylinder of the rod stock, as shown in FIG. 3. Definition of the slot 14 forms the internal surface of the base portion which, in FIG. 3's implementation, also has the shape of an arc. For example, the arc may a portion of a circle with a center 0.082 inch from the axis of the arms 12 and having a 0.063 inch radius. Extending between the external and internal surfaces are 0.010 inch rounds on a center offset 0.039 inch and 0.030 inch from the axis of the arms 12. The net effect of these surfaces is to smooth any edges that might snag or catch the renal artery during or after implanting. Also, these surfaces give the base portion 18 a generally (but not mathematically precise) elliptical shape in the cross-section of FIG. 3.

The slot 14 may have a range of widths, depending upon the degree of hypertension desired and/or the size of the animal. For example, widths of 0.20, 0.23, 0.25, 0.27, 0.30 and 0.35 mm at depths of 2 mm may be formed. Examples of saws used to cut the slot 14 include solid carbide saws with a ½ inch diameter hub, 20 teeth and ¾×0.009×¼ or ¾×0.0098×¼ or ¾×0.0106×¼ dimensions, such as those offered by RobbRJJack Corporation of Lincoln, Calif.

The slot 14 of the illustrated implementation is radial and non-concentric and has a depth of 0.079 inch from the thickest radial component of the cross-section of the base portion 18. Thus, the minimum depth of the slot 14 is roughly 70% of the diameter of the rod stock. The depth increases as the base portion 18 tapers at its ends. An advantage of the illustrated shape of the negative space of the slot 14 (and the shape of the base portion 18) is that it can be efficiently constructed by reciprocating the rod stock on its long axis while the saw forming the slot 14 removes material from the rod.

The suture openings 16 are positioned near the opposite radial edge (for the rat implementation about 0.046 inch from center) of the arms from the base portion 18 and are cylindrical passages defined between the lateral and medial surfaces of the arms, as shown in FIGS. 2 and 3. The diameter of the cylindrical passages may be sized to accommodate expected suture sizes, such as with a 0.010 inch or 0.254 mm diameter. They're also axially aligned to facilitate passage of a suture therethrough during implanting of the clip 10. This advantageously locks the clip onto the renal artery so that it does not come loose, which the prior art clips are prone to do. Other openings with different size, shape, placement or structure, such as posts, could be formed to retain the sutures, but smooth coaxial bores are easily threaded with suture in surgical settings.

It should be noted that other closure mechanisms may be employed, such as a clip or latch that swings closed once the arms 12 are extended around the renal artery. Some type of spring-loaded post could also be employed. However, the suture openings are preferred for simplicity and work well with surgeons familiar with sutures.

Implementations of the clip 10 may have a range of advantages. For example, conventional clips only produce hypertension in 40-70% of animals meaning that a lot of animals are used without any relevant data being produced. The illustrated clip implementation has produced hypertension in over 95% or 96% of rats with an n=26 study size. The uniform constriction from the slot 14 produces a very reliable and reproducible level of hypertension. Also, because of the retaining mechanism, the clip remains in the desired position within the animal yielding these improved results. Higher reliability saves significant costs when animal models are employed.

In another study, application of these clips 10 to the left renal artery produced reliable and consistent levels of hypertension in rats. Nine day application of clips 10 with gap widths of 0.27, 0.25, and 0.23 mm elicited higher mean arterial blood pressures of 112±4, 121±6, and 135±7 mmHg, respectively (n=8 for each group) than those of sham-operated controls (95±2 mmHg, n=8). Moreover, 8 out of 8 rats in each of the 0.23 and 0.25 mm clipped group were hypertensive whereas 7 out of 8 rats in the 0.27 mm clipped group were hypertensive. Plasma renin concentrations were also increased in all clipped groups as compared to sham-operated controls. Rats with renal clips of widths 0.27, 0.25 or 0.23 mm had significantly higher heart rates (392.5±7.1, 373.0±12.7, and 395.3±6.1 beats per minute, respectively) compared to sham-operated rats (343.3±14.3 beats per minute).

A number of aspects of the systems, devices and methods have been described. Nevertheless, it will be understood that various modifications may be made without departing from the spirit and scope of the disclosure. For example, clips of varying size can be produced by varying the size of the cleft where the renal artery sits, such that different levels of hypertension are produced. And, the clip design is scalable so that it could be adapted for different animals, such as mice, dogs, pigs, sheep or primates. Accordingly, other aspects are within the scope of the following claims.

That which is claimed:

1. A vascular clip comprising:
a pair of arms defining a slot extending therebetween;
a base portion connecting the pair of arms; and
at least one curved surface.

2. The vascular clip of claim 1, wherein the base portion defines an end portion of the slot.

3. The vascular clip of claim 2, wherein the base portion includes the curved surface and the curved surface defines the end portion of the slot.

4. The vascular clip of claim 3, wherein the pair of arms extend parallel to each other from the base portion.

5. The vascular clip of claim 4, wherein the pair of arms have a curved peripheral surface.

6. The vascular clip of claim 5, wherein the base portion extends between ends of the pair of arms and the clip has a U-shape.

7. The vascular clip of claim 6, wherein the base portion has an elliptical cross-section.

8. The vascular clip of claim 7, wherein the curved peripheral surface is a cylindrical surface.

9. The vascular clip of claim 7, wherein the curved peripheral surface extends over a portion of the base portion not defining the slot.

10. The vascular clip of claim 1, wherein at least one of the pair of arms includes the curved surface.

11. The vascular clip of claim 10, wherein the curved surface includes a medial bevel defining a peripheral edge of the slot.

12. The vascular clip of claim 11, wherein the curved surface includes a lateral bevel extending about lateral edges of the arms.

13. The vascular clip of claim 1, wherein the vascular clip is of unitary construction and all intersecting surfaces form oblique angles.

14. The vascular clip of claim 1, further comprising a slot closure mechanism.

15. The vascular clip of claim 14, wherein the slot closure mechanism includes a suture retainer.

16. The vascular clip of claim 15, wherein the suture retainer is positioned on an end of the slot opposite the base portion.

17. The vascular clip of claim 16, wherein the suture retainer includes a pair of openings defined by the pair of arms.

18. The vascular clip of claim 17, wherein the pair of openings are aligned along an axis.

19. The vascular clip of claim 18, wherein each of the openings has a medial end and a lateral end.

20. The vascular clip of claim 19, wherein the medial end communicates with the slot.

21. The vascular clip of claim 20, wherein the openings have a cylindrical shape.

22. The vascular clip of claim 1, wherein the arms and base portion are at least partially comprised of a material with a Young's modulus greater than 83 GPa.

23. The vascular clip of claim 22, wherein the material has a Young's modulus of 100-110 GPa.

24. The vascular clip of claim 23, wherein the vascular clip is comprised of titanium.

25. A vascular clip comprising:
a pair of arms defining a slot extending therebetween;
a base portion connecting the pair of arms; and
a slot closure mechanism.

26. The vascular clip of claim 25, wherein the slot closure mechanism includes a suture retainer.

27. The vascular clip of claim 26, wherein the suture retainer is positioned near an end of the slot opposite the base portion.

28. The vascular clip of claim 27, wherein the suture retainer includes a pair of openings defined by the pair of arms.

29. The vascular clip of claim 28, wherein each of the openings has a medial end and a lateral end.

30. The vascular clip of claim 29, wherein the medial end communicates with the slot.

31. The vascular clip of claim 30, wherein the pair of openings are aligned along an axis.

32. The vascular clip of claim 31, wherein the openings have a curved internal surface.

33. A vascular clip comprising:
a pair of arms defining a slot therebetween; and
a base portion connecting the pair of arms;
wherein the arms and base portion are at least partially comprised of a material with a Young's modulus greater than 83 GPa.
